(19) United States
(12) Patent Application Publication
(10) Pub. No.: US 2018/0071117 A1
(43) Pub. Date: Mar. 15, 2018

(54) PROSTHETIC VALVE AND ASSEMBLY FOR AMPUTEES AND METHODS OF USE

(71) Applicant: ENGINEERED SILICONE PRODUCTS, LLC, Newton, NJ (US)

(72) Inventors: Louis James Haberman, Denville, NJ (US); Robert Pietrowicz, Rockaway, NJ (US)

(21) Appl. No.: 15/701,283

(22) Filed: Sep. 11, 2017

Related U.S. Application Data
(60) Provisional application No. 62/385,710, filed on Sep. 9, 2016.

(51) Int. Cl. A61F 2/80

(52) U.S. Cl. CPC A61F 2/80; A61F 2002/7856; A61F 2002/805

(57) ABSTRACT

A one-way air expulsion plug device and system are provided, which is manually inserted into a valve body, wherein the device and/or system provides the user or wearer with actual examination and/or palpitation of a distal limb requiring the device and/or system with a prosthetic limb or attachment and is in a configuration that can tolerate an elevated negative atmospheric socket suspension system.

### CROSS-REFERENCE TO RELATED APPLICATIONS

This application claims priority to of U.S. Provisional Application No. 62/385,710 filed Sep. 9, 2016, the contents of which is incorporated by reference herein in its entirety.

### FIELD

The present invention relates to the field of prosthetics and supporting devices for amputees.

### BACKGROUND

Prosthetics are often suspended to the amputated limb (residuum) a prosthetic socket by various means. Prosthetics can be held in place in a number of ways, including with shuttle lock systems, suction systems, and vacuum systems. Prosthetics can be designed with a specified level of activity in mind. Prosthetics designed for greater activity require greater attention to detail when fitting the prosthetic to a limb. One way of achieving a snug, consistent, and reliable fit for the enhanced suspension of a prosthesis is by donning the prosthetic limb and then integrate either a standard static suction (e.g., 0 atmosphere) system or a high negative vacuum (−1 to −23 in. Hg) system between a prosthetic inner socket and the amputated limb, thereby forming an integral connection and an air-tight seal between the two. Prior art systems for achieving this vacuum or suction are inadequate and improvements are needed. Embodiments of the inventions described herein solve problems of the prior art and, among other things, allow for the improved fitting, ease of use and unrestrictive diurnal application of a prosthetic by the user without the need of intervention from a medical professional, such as a prosthetist, to help maintain functionality of current, less robust and less reliable valve systems.

### DESCRIPTION OF DRAWINGS

FIG. 1 is a view of one embodiment of the present invention showing a rotatable, quick-disconnect and screwable, vacuum valve plug assembly with attached hose, a valve body and valve housing for prosthetics.

FIG. 2 is a side view of the components of FIG. 1 wherein the components are mated to form one embodiment of a system of the present invention.

FIG. 3 is a top view of FIG. 2 providing a different perspective of an embodiment of a system of the invention.

FIG. 4 is a view of the components of FIG. 1 with a view of a rotatable, quick-disconnect and screwable, high-vacuum valve plug assembly that is detached from a valve body that is detached from a valve housing.

FIG. 5 is a view of the components of the FIG. 4 embodiment with a further breakdown of components for an embodiment of a prosthetic valve.

FIG. 6 is a view of an embodiment of a prosthetic valve as described herein in operation in conjunction with a prosthetic limb.

FIG. 7 is a view of a prosthetic socket with an inner valve housing secured to an outer socket using a locking ring attached thereto, with the prosthetic valve body and valve stem removed.

FIG. 8 is an alternative embodiment of a barb portion.

FIG. 9 is an alternative embodiment of a prosthetic valve showing a blown-up diagram of the parts of a prosthetic valve.

### DETAILED DESCRIPTION

Embodiments of a prosthetic valve as described herein may comprise a valve plug assembly and housing comprising: a valve housing comprising: a valve body; and a locking ring; a valve plug assembly comprising: a finger grip portion; a barb; a cylindrical portion protruding from the finger grip portion wherein the cylindrical portion is configured to fit inside of and create an airtight seal with the valve body; and at least one o-ring. In some embodiments a valve plug assembly may comprise an upper o-ring; and/or a lower o-ring. In some embodiments, a barb can be a 360 degree rotatable barb, wherein the barb is rotatable relative to a finger grip portion of a valve plug assembly. In some embodiments a barb can be a 90 degree barb, a 45 degree barb, or other angle, which is not particularly limited and such angles would be envisaged by the skilled artisan. A finger grip portion may be circular along its outer diameter. In some embodiments, a finger grip portion is not hexagonal.

An embodiment of the present invention comprises a one-way air expulsion plug device that can be manually inserted into a valve body, wherein the valve body can be a plastic or similar material. In some embodiments, the invention comprises a double O-ring stem that can rotate, a larger circumference top, a 360 degree rotatable barb that can connect via a hose, preferably a flexible hose, to an external vacuum system, electronic or manual, for suspending a prosthesis by, for example, evacuating air, attaching, and/or sealing a prosthetic limb to an amputee for various amputation levels. In certain embodiments, the invention comprises a stem portion that mates with the valve body. In other embodiments, a stem portion comprises a snap mechanism or a click mechanism for mating with the valve body. Embodiments of the invention comprising a one-way air expulsion plug device provide the unexpectedly superior results of being able to expel air during pumping, while also allowing for pushing-in and pulling-out the O-ring stem using finger pressure. Certain embodiments utilize a valve housing, which can be a plastic or similar material and can be molded or otherwise attached to a prosthetic socket. In some embodiments, a valve housing can be threaded for mating with the valve body. In other embodiments, the valve body can either reversibly mate with the threaded valve housing or irreversibly mate with the valve housing. The configuration of the attachment (e.g., threading) is not particularly limited.

In certain embodiments of the present invention, the 360 degree rotatable barb and stem can provide the unexpected results of eliminating torqueing and twisting of the housing and allowing for inspection or actual examination of the distal limb without removal of the prosthetic. Other embodiments of the present invention comprise systems with the various combinations of components described herein for maintaining a suction seal throughout a 360 degree rotation, with no hose binding, for amputees. The systems described herein can comprise and/or function with various pumping systems, such as, for example, any pumping system (e.g., electronic or mechanical) for elevated vacuum suspension-type sockets, including simple hand pumping systems.

In some embodiments and methods, a valve body of the present invention can be retrofitted to an existing valve housing previously attached to a prosthetic limb. In further embodiments, the product and systems comprise a valve body that can be unscrewed or unmated from a valve housing to allow for examination or palpation of a distal limb of an amputee. In some embodiments, the valve body can be screwed into the valve housing, allowing for stem rotation and compressing at least one o-ring that can form a seal. As will be appreciated, certain embodiments allow for convenient placement of the valve housing for a patient and/or individual tasked with aiding or caring for an amputee. Embodiments of the present invention also provide the superior, unexpected results by avoiding and/or preventing distal limb edema by allowing for an improved and superior seal and physical examination and positioning of a distal limb in a socket of a prosthetic limb, which can be used to confirm that total contact is present.

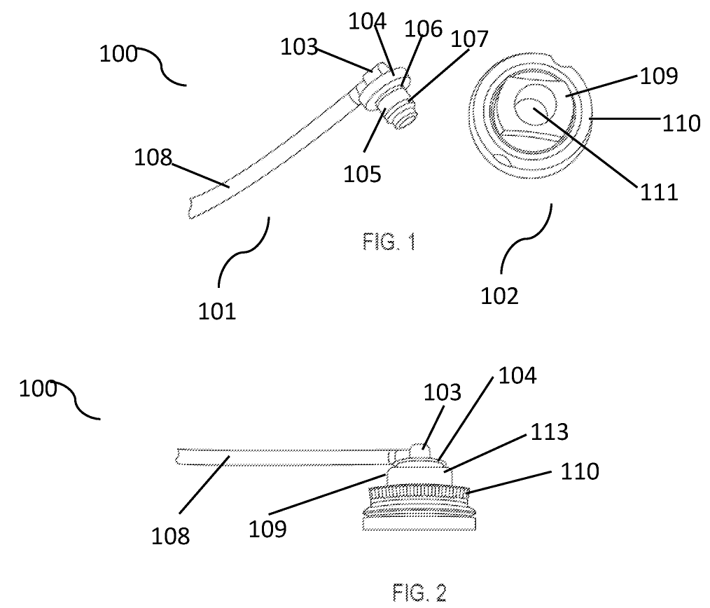

The components and systems described herein can be made of various materials capable of forming similar structures and functions as those described herein and shown in the figures. The figures provide certain aspects of the invention and provide reasonable, though not limiting, methods of constructing and using the described and illustrated embodiments. The devices and/or systems of the present invention are not limited to any particular distal limb amputation or undeveloped limb or limb bud. The devices and or systems of the present invention can be deployed in various positions and/or multiple positions depending on the needs of the individual and/or prosthetic and/or limb amputation and these positions and configurations would be recognizable to one of skill in the art. Moreover, the devices and systems of the present invention are not limited in size or caliber of materials or final products, and the individuals needs will dictate the necessary sizes, calibers, materials, etc. FIG. 1 shows an example of a prosthetic valve assembly 100 with a valve plug assembly 101 and a valve housing 102 with valve plug assembly 101 detached from the valve housing 102. The valve plug assembly 101 can comprise a barb 103 connected to a finger grip portion 104 that can comprise a stem 105. The stem 105 can include an upper o-ring 106 and a lower o-ring 107. Stem 105 can house a bleeder valve, such as a duck-bill valve, or other one-way valve configured to allow air to be drawn out of a prosthetic, while preventing return or infiltrating air and creating and maintaining a seal and/or vacuum when installed into a prosthetic socket and connected by a hose to a vacuum source. Embodiments comprising such a one-way valve can produce the surprising, unexpected, and improved results of being able to sustain and withstand the high negative pressure exerted by a vacuum pump, which can be greater than zero atmospheric suction.

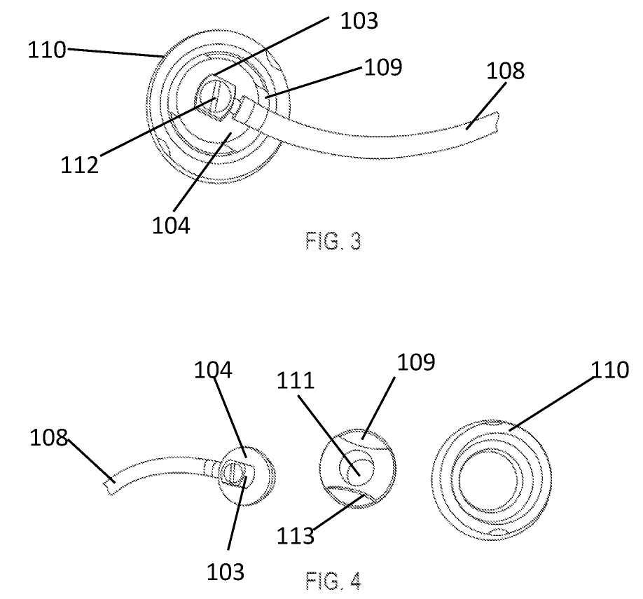

Barb 103 can be configured to attach to a hose 108 and is configured to create an airtight seal with the hose. As shown in FIG. 3, barb 103 can also comprise a fastener 112, such as but not limited to a screw, that can secure barb 103 to finger grip portion 104. In some embodiments, barb 103 itself can be threaded to be threadedly secured to finger grip portion 104. In some embodiments, hose 108 and barb 103 can be made as a single, integral unit. Hose 108 can be detachable from barb 103. Hose 108 may be polyethylene, polyurethane, vinyl, polyvinyl chloride, and the like. A valve assembly 100 can comprise a valve housing 102 comprising a valve body 109, locking ring 110 to secure the valve housing to a prosthetic socket and a valve body opening 111 to easily accept stem 105. Locking ring 110 can be aluminum, titanium, stainless steel, and the like.

As shown in FIG. 4, valve body 109 can have indented and/or flattened portions around valve body opening 111 that can be shaped to provide a contact surface 113 can aid with fingertip control and to assist in securing the valve body to a valve housing 110. The shape of the contact surface 113 is not particularly limited, but it can be shaped to have flat portions that are configured to provide a surface to aid in threading the valve body into a valve housing or snapping a valve body into a valve housing, or configured to mate with a tool such as a wrench to secure the valve body in a valve housing.

An example of a suitable inner diameter of a valve housing, for transfemoral amputees, is about 26 mm. A valve housing diameter can also be about 20-30 mm, about 25-30, mm, about 20-25 mm, and ranges therebetween. An example of a valve body height is about 19 mm, which can be configured so as not to protrude far beyond an outer socket contour of a prosthetic device. A valve body height may be about 15-20 mm, about 15-25 mm, and ranges therebetween. An example of a diameter of a fingergrip portion is about 21 mm, which can be sized to permit a user easy fingertip access and security for a stem's installation and removal even for users with hand impairment. The fingergrip portion's size is not particularly limited and can have a diameter of about 20-25 mm, about 15-25 mm, about 20-22 mm, and ranges therebetween. An example of a stem can have an about 11 mm long by 12 mm thick cylindrical barrel that may accommodate 1, 2 or 3 O-rings for a positive seal to the valve body. In some embodiments, a stem can have a thickness of about 10-12 mm, about 10-13 mm, about 10-15 mm, about 12-15 mm, and ranges therebetween. A stem can have a length of about 10-15 mm, about 12-15 mm, about 10-13 mm, and ranges therebetween. A stem may also house a bore hole of about at least 1 mm through the cylindrical barrel of a stem to enable air to be evacuated from a prosthetic socket. The stem bore may be about 0.5-1.5 mm, about 0.75-1.25 mm, about 1-1.5 mm, and ranges therebetween. A stem may also comprise a tapered lower section that is about 2.8 mm, configured to facilitate user placement of the stem into the valve body for proper seating. The tapered lower section can be about 2-3 mm, about 2.5-3 mm, about 2.75-3 mm, about 2.75-3.25 mm, about 3-3.25, mm, and ranges therebetween. A stem and valve body can be configures so that an auditory click is heard to provide confirmation for the user that the stem and the valve body are fully engaged and forming an airtight seal. A fingergrip portion of a valve plug assembly can be about 21.5 mm in diameter, which encourages ease of use via simple prehension. The entire prosthetic valve assembly 100 can be made proportionately smaller to fit the needs of children, adolescents and smaller adults, and can be suitably configured for other limbs, and such sizing would be immediately envisaged by the skilled artisan.

In some embodiments, a valve body comprises an o-ring around its outer circumference. In some embodiments a valve body can be configured so as to form an airtight seal when finger pressure is applied to screw the valve body into an inner circumference of a locking ring. Referring to FIGS. 1-5, stem 105 and corresponding at least one o-ring can be sized to fit inside of the valve body opening 111 to create an airtight seal to allow for forming a vacuum for attaching and securing a prosthetic limb to the distal end of an amputee's limb. Stem 105 can comprise polyoxymethylene, polyethylene, polypropylene, polyurethane, polyvinyl chloride, and the like, and mixtures thereof. A stem can be formed by machining, casting, 3D printing, molding, and the like. In some embodiments, a stem is made by machining a single cylinder of material comprising, for example and without limitation, polyoxymethylene, thus creating a finger grip portion having a circumference larger at a first end that is configured to mate with a finger grip portion of a valve plug and a smaller circumference at a second end. In some embodiments, the smaller circumference portion can have a diameter that is 95%, 90%, 85%, 80%, 75%, 70%, 65%, 60%, 55%, 50%, 45%, 40% of the diameter of the first end with the larger circumference. A stem may comprise detents or other aspects that allow it to form an airtight seal and to grip and/or hold a valve body opening when inserted into the valve body opening, while also allowing the stem to be removed from and inserted into a valve body with only finger pressure. A stem may also be made so that it has at least one indentation that retains and holds in place at least one o-ring, which can allow for the repeated removal and insertion of a stem into and out of a valve body opening. This configuration can produce the surprising and unexpected results of allowing a user to repeatedly and reliably form a vacuum in a prosthetic using only finger pressure when inserting the stem into a valve body opening.

FIG. 2 shows an example of a prosthetic valve assembly 100 with valve plug assembly 101 inserted into valve housing 102. Valve plug assembly 101 can be inserted into valve housing 102 so that a flat portion of finger grip portion 104 is flush against the upper surface of valve body 109, forming an airtight seal that allows for air to be evacuated from a prosthetic that has the valve housing 102 installed into it. Locking ring 110 may be reeded, knurled, or otherwise textured along its outer circumference to allow it to be gripped for screwing into or otherwise connecting to a prosthetic. Locking ring 110 may be threaded along its lower portion of its outer circumference so as to be securely connected to a prosthetic socket by a prosthetist. Valve housing 102 and all or some of its components may be installed and secured with an adhesive or it may be directly molded into the prosthetic or integrally formed into a prosthetic socket, which can be accomplished by using a disposable molding dummy.

Providing two o-rings on a stem can provide for an improved seal that allows a valve plug assembly to form an airtight seal to a valve body while also being removable with only finger pressure. Such an arrangement provides for the improved and unexpected results to be able to form and maintain a seal when air is evacuated at negative pressures between about −1 to about −25 inches of mercury (in. Hg.) while also being removable with only finger pressure. Negative pressures can be about −1 to about −10 in. Hg, about −10 to about −20 in. Hg., about −15 to about −25 in. Hg., about −10 to about −25 in. Hg, and ranges therebetween. Lower limb amputees can generally tolerate (and, at times, prefer) a higher negative atmosphere while upper limb amputees prefer (and, at times, require) a lesser negative atmosphere. These differences may be due to the cross-sectional dimensional differences of upper and lower residuums (smaller vs larger surface areas), as well as internal limb hypersensitiveness such as neuromas and adhesions and/or scarred tissues. Users can set the chosen pump device to acquire and maintain very specific negative atmospheric values that are comfortable yet will also properly suspend the prosthesis, and these corresponding pressures would be immediately understood by the skilled artisan given the context and the particular application. A valve assembly as described herein can be configures to convey any of these amount of negative atmosphere that are desired. Such an arrangement can allow for increased ease of use for an amputee to more easily, more efficiently, and more effectively install a prosthetic without the need for intervention from a medical professional, such as a prosthetist, to reestablish a positive seal. Embodiments of the invention described herein provide the improved results of greatly improving the installation of prosthetic limbs that are designed for both normal and even greater amounts of activity, thus allowing amputees to increase their mobility and dexterity for a prosthetic attached to a lost limb.

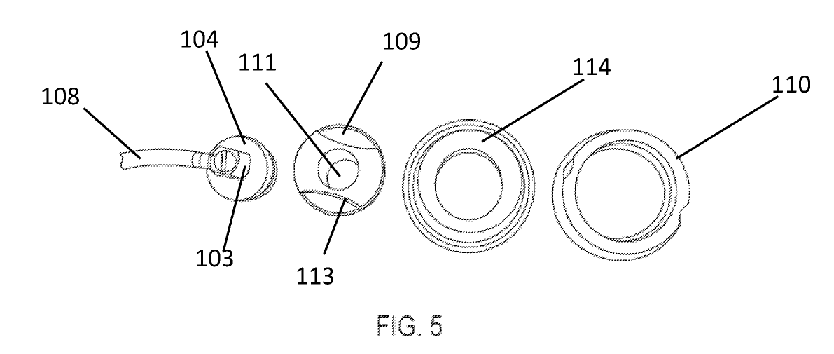

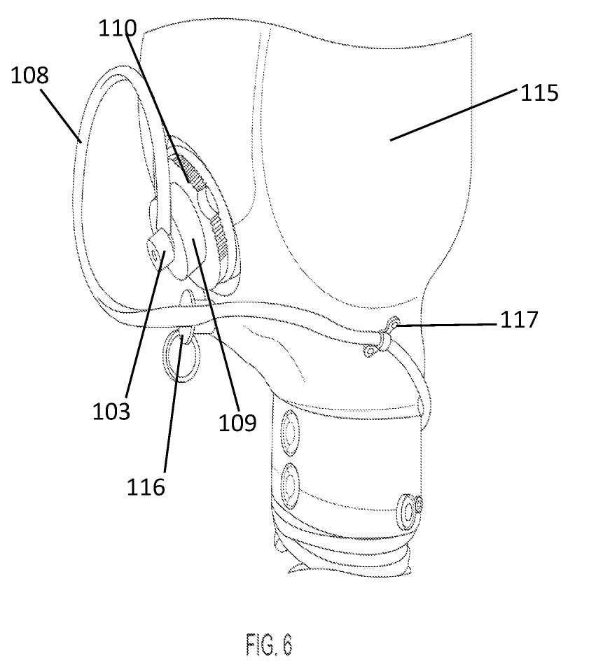

Valve body 109 can be threaded so that it can be threadedly attached to locking ring 110. A locking ring can be threaded on its inner circumference so that it may securely mate with a valve body. Referring to FIG. 5, locking ring 110 may comprise an inner ring portion 114 that can be removable from the locking ring. The inner circumference of locking ring 110 may be threaded to mate with threading along the outer circumference of inner ring portion 114. Inner ring portion 114 can be threaded on its inner circumference to mate with valve body 109. Valve body 109 may have threading to secure to the inner circumference of inner ring portion 114. Valve body 109 and inner ring portion 114 can be securely attached to one another using threading, twist-lock mechanisms, ¼ turn lock mechanisms, snapping mechanisms, and the like. A valve housing can be formed as a singular unit comprising a valve body. A valve housing can be formed as a single unit with an inner ring portion, where inner ring portion is not separable from the valve housing. In some embodiments, a valve housing can be formed as a single unit with an inner o-ring portion that is separable from the valve housing and can be replaced by a prosthetist, on-site. Prosthetic valve assemblies described herein can be fully cleaned, repaired and serviced, on-site by a prosthetist. In some embodiments, locking ring 110 and inner ring portion 114 can be made of the same or different materials. In some embodiments, inner ring portion 114 can comprise aluminum, polyoxymethylene, polyethylene, polypropylene, polyurethane, polyvinyl chloride, and the like, and mixtures thereof. A valve body 109 may be attached to a locking ring and/or inner ring portion with threads, teeth, clamping, one-touch fastening, ¼ turn fastening, and the like.

In some embodiments, a locking ring may be made of a material such as aluminum and can comprise internal threads along its inner circumference that can comprise polyoxymethylene, polyethylene, polypropylene, polyurethane, polyvinyl chloride, and the like, and mixtures thereof. A valve body can comprise aluminum, titanium, stainless steel, polyoxymethylene, polyethylene, polypropylene, polyurethane, polyvinyl chloride, and the like, and mixtures thereof. Embodiments of a prosthetic valve assembly as described herein provide for disassembly of individual pieces while also allowing for reassembly of the device without any special tools, or the use of hand force or finger pressure only. Such an arrangement provides for a device having improved hygienic properties because it can easily be disassembled, cleaned, and reassembled and surprisingly also produce a proper seal to achieve a vacuum when installing a prosthetic limb.

Some embodiments of the invention described herein comprise a prosthetic socket comprising a locking ring and an air evacuation valve. The locking ring can be configured as described herein to mate and secure with a valve body and valve plug assembly as described herein. Embodiments of a prosthetic socket can comprise a valve housing that can be externally and internally threaded to secure a valve body and a locking ring, and an air evacuation valve. A valve assembly can be attached to a prosthetic device and a pump and can be configured to permit a high and rapid passage of air to achieve a high negative vacuum atmosphere when air is evacuated after attaching a valve plug assembly such as those described herein.

Certain embodiments comprise a method of securing a prosthetic device to an amputated limb comprising attaching a prosthetic limb to the end of an amputated limb, attaching a locking ring to an opening and connecting to a socket in the prosthetic limb; attaching a valve body to the locking ring; attaching a valve plug assembly to the valve body; attaching a hose to the valve plug assembly; attaching a vacuum pump to the hose; applying a vacuum to the hose to apply a vacuum to the prosthetic limb; and creating a vacuum-tight seal of the prosthetic limb to the amputated limb. In some embodiments, the valve plug assembly is attached to valve body without the use of threading. In some embodiments, a valve body is attached to a valve housing without any threading. In some embodiments, a valve plug assembly is attached to a valve body without threading and the valve body is attached to the housing with threading.

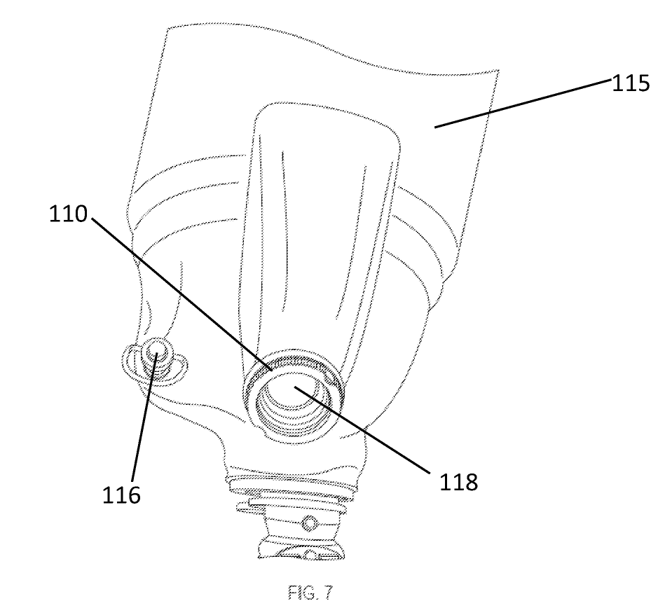

FIGS. 6-7 show an embodiment of affixing a prosthetic valve to a prosthetic. FIG. 6 shows an attached prosthetic valve comprising a locking ring 110, hose 108, barb 103 and valve body 109 attached to a prosthetic 115. As shown by FIG. 6, hose 108 can also be attached to a prosthetic 115 using a hose clip 117. Some embodiments may not comprise a hose clip. FIG. 7 shows a prosthetic 115 with a prosthetic valve removed but with a locking ring 110 still in place exposing a prosthetic opening 118.

In some embodiments, a prosthetic valve may comprise a valve plug assembly, a valve body, a valve housing, and a hose attached to the valve plug assembly; wherein the prosthetic valve is attached to a prosthetic and the prosthetic valve remains attached to the prosthetic while the prosthetic is in use. In some embodiments, after applying a vacuum using a prosthetic valve as disclosed herein, a valve plug assembly may be removed and a plug may be inserted into a valve body opening, thereby retaining a vacuum inside of the prosthetic.

In some embodiments, a method of securing a prosthetic device to an amputated limb can comprise attaching a liner to an amputated limb prior to attaching the prosthetic device. A liner can comprise fabric, silicone gel, urethane or the like and combinations thereof. A liner can be used to in conjunction with a prosthetic, which can improve the vacuum and ease the stresses to the skin in the presence of standard suction suspension or elevated vacuum suspension that can be applied to a prosthetic limb when securing the prosthetic to the limb.

Valve body 109 can be configured so that it is absent any air holes in its flat bottom aspect, which may not make contact with, either, the liner over the residuum or the bare skin if no liner is present. Thus, air may not be exhausted through such holes, which can be common in other valves of the prior art, which can cause skin blisters and/or damage to a polymer liner. Embodiments of the invention described herein can exhaust air around the outer perimeter of a valve body and/or an inner perimeter of an inner surface of a valve housing. This is an additional unique feature of this Valve Assembly. Certain embodiments as described herein can comprise a circular venting system with a clearance of about 1/5000^{th}'s of an inch. The sizing of a circular venting system can be configured so that it is too narrow for skin tissue to enter into but large enough to permit the easy flow of air for evacuation.

Embodiments of attaching a prosthetic limb to the distal end of an amputated limb can produce the unexpected and improved results of reducing unwanted movement of the prosthetic relative to the amputated limb, which provides for reduced injuries to a patient. Such injuries can include but are not limited to, edema, skin injury resulting from shear, rotation, or friction, blisters, sores, and the like. Accordingly, embodiments of the devices and methods as described herein provide for a way of reducing or eliminating these injuries to an amputated limb. Embodiments of the devices described herein can create an improved seal between a prosthetic and an amputated limb, thus improving a user's proprioception in a drastic improvement over other prior art prosthetics. Embodiments of the methods and devices described herein can allow for an improved seal between a prosthetic and an amputated limb and can maintain an improved seal as a limb swells and shrinks, diurnally as a result of changes in body temperature, blood flow, salt retention and consumption of certain foods and drinks, etc. By providing for an improved seal that accounts for such swelling and shrinking, the methods and devices described herein can achieve the improved and unexpected results of maintaining an improved seal for 12, 24, 36, 48 hours, or longer.

Certain embodiments comprise a method of making a prosthetic socket comprising placing a valve housing in an opening in a prosthetic socket so that a portion of the valve housing passes through the opening attaching a locking ring to the opening in the prosthetic socket; attaching a valve body to the locking ring; attaching a valve plug assembly to the valve body.

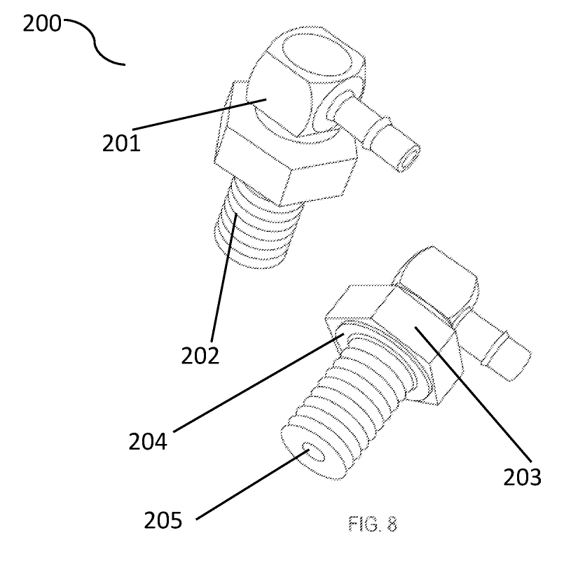

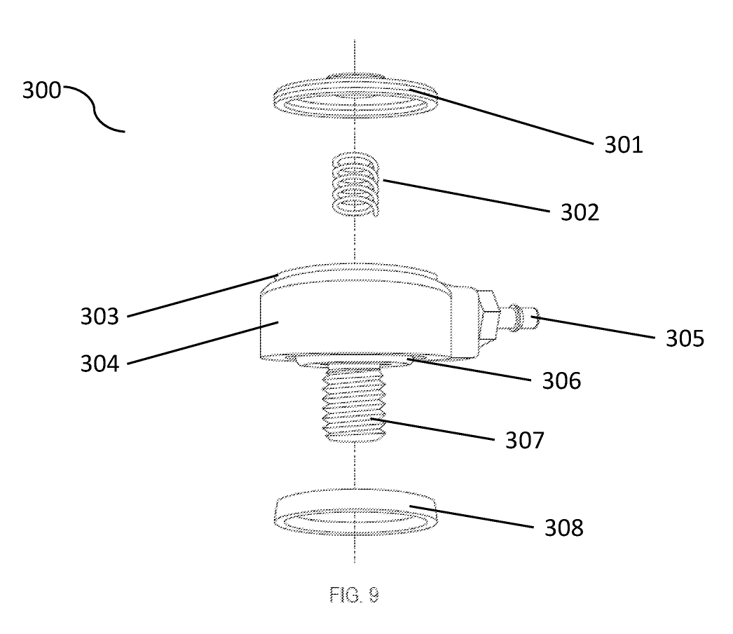

FIG. 8 shows an alternative embodiment of a barb portion 200. Barb 201 can comprise threading 202, barb body 203, at least one o-ring 204, and air hole 205. Embodiments of barb portion 200 can be used with a valve body and locking ring as described herein. In some embodiments using a barb portion 200 comprising threading 202, a valve body opening can comprise threading to mate with threading 202. Barb body 203 can be configured to have a hexagonal shape configured for interaction with a wrench to ensure an airtight fit when a barb portion 200 is installed into a prosthetic socket when it is tapped to receive the threaded section (e.g., 5/16″×18). Embodiments having barb 200 may be used independently of valve body 109, an can be used as a stand-alone air evacuation system. A non-air-exhausting valve body may be used in conjunction with barb 200. In some embodiments, barb portion 200 may be tightened using only finger pressure and threading 202 and at least one o-ring 204 can be configured to provide an air tight seal. Barb portion 200 comprising barb 201 can allow for 360 degree rotation of barb 201 relative to barb body 203, which can produce the described superior results of allowing a hose to remain attached to barb 201 when a prosthetic valve is attached to and forming a vacuum seal for a prosthetic.

In an alternative embodiment, a prosthetic valve assembly can comprise a pressure-release portion; a valve body portion that is connected to the pressure-release portion wherein the valve body portion comprises a stem and a barb; wherein the stem can be threaded; and the valve body can comprise a valve diaphragm and a valve spring. The prosthetic valve assembly may also comprise a socket nut that can be threaded to mate with a threaded stem and secure the prosthetic valve assembly to a socket of a prosthetic. The valve body may comprise an internal vent spool. FIG. 9 shows a blown-up view of such an embodiment comprising prosthetic valve 300 comprising diaphragm 301, spring 302, spool assembly 303, valve body 304, barb 305, at least one o-ring 306, threaded stem 307, and valve nut 308. In certain embodiments, prosthetic valve 300 can fit directly over an opening in a prosthetic with threaded stem 307 fitting through a prosthetic opening wherein valve nut 308 can comprise a threaded opening that can be attached and secured to the threaded stem 307 to compress at least one o-ring 306 against a prosthetic to create an air tight seal when air is vacuumed out of the prosthetic by applying a vacuum pump to barb 305, which can vacuum air through threaded stem 307, and barb 305, which can be attached to a hose that is connected to the vacuum pump. In some embodiments, prosthetic valve 300 may be inserted into a valve body opening as described herein. In some embodiments, a valve body can have mating threads to be secured to valve stem 307. In some embodiments, threaded stem may be configured as a stem with a single or double o-ring stem that can be inserted into a valve body opening with finger pressure and form an airtight seal as set forth herein.

As used herein the terms, “prosthetic”, “prosthetic device”, and “prosthetic limb” can be used interchangeable and are not particularly limiting. The skilled artisan would understand the meaning of these terms to include prosthetic arms and legs and variations of these.

As used herein, the terms “finger pressure” and “hand force” can be used interchangeably and refer to the amount of force needed to manipulate (e.g., remove or insert) portions of the embodiments as described herein and the meaning of which would immediately be understood by the skilled artisan. For example, and without limitation, finger pressure may refer to the amount of force that is applied to remove one part of a prosthetic valve as described herein, where such force can be applied without the aid of tools.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. A prosthetic valve assembly comprising:
a valve housing comprising:
a valve body; and
a locking ring;
a valve plug assembly comprising:
a finger grip portion;
a barb;
a cylindrical portion protruding from the finger grip portion wherein the cylindrical portion is configured to fit inside of and create an airtight seal with the valve body;
and at least one o-ring.

2. The prosthetic valve assembly of claim 1, further comprising a hose connected to the barb.

3. The prosthetic valve assembly of claim 1, wherein the barb is a 90-degree barb.

4. The prosthetic valve assembly of claim 1, wherein the valve housing further comprises an inner ring portion.

5. The prosthetic valve assembly of claim 1, wherein the cylindrical portion protruding from the finger grip portion forms an airtight seal with a valve body opening of the valve body.

6. The prosthetic valve assembly of claim 5, wherein the valve plug assembly is configured to form an airtight seal with the valve body by only applying finger pressure to fit the valve plug assembly into the valve body.

7. The prosthetic valve assembly of claim 1, wherein the at least one o-ring comprises an upper o-ring and a lower o-ring.

8. A valve plug assembly for applying a vacuum to a comprising:
a barb;
a finger grip portion;
a stem wherein the stem comprises:
an upper o-ring and a lower o-ring.

9. The valve plug assembly of claim 8, wherein the barb is a 360 degree rotatable barb.

10. The valve plug assembly of claim 8, wherein the stem comprises a material that is selected form the group consisting of polyoxymethylene, polyethylene, polypropylene, polyurethane, polyvinyl chloride, and mixtures thereof.

11. The valve plug assembly of claim 8, wherein the finger grip portion and the stem are formed by machining a single piece of polyoxymethylene.

12. A method of securing a prosthetic device to an amputated limb comprising the steps of:
attaching a prosthetic socket to the end of an amputated limb;
placing a valve housing in an opening in the prosthetic socket so that a portion of the valve housing passes through the opening
attaching a locking ring to the opening in the prosthetic socket;
attaching a valve body to the locking ring;
attaching a valve plug assembly to the valve body;
attaching a hose to the valve plug assembly;
attaching a vacuum pump to the hose;
applying a vacuum to the hose to apply a vacuum to the prosthetic limb; and
creating a vacuum-tight seal of the prosthetic limb to the amputated limb.

13. The method of claim 12, wherein the valve plug assembly is attached to the valve body without the use of threading.

14. The method of claim 12, wherein the valve body is attached to the valve housing without any threading.

15. The method of claim 12, wherein the valve plug assembly is attached to the valve body without threading and the valve body is attached to the housing with threading.

16. The method of claim 12, wherein the valve body is attached to the valve housing with threading.

17. A prosthetic socket comprising the prosthetic valve of claim 1.

18. A method of making a prosthetic socket comprising placing a valve housing in an opening in a prosthetic socket so that a portion of the valve housing passes through the opening
attaching a locking ring to the opening in the prosthetic socket;
attaching a valve body to the locking ring;
attaching a valve plug assembly to the valve body.
